(19) United States
(12) Patent Application Publication
(10) Pub. No.: US 2021/0398608 A1
(43) Pub. Date: Dec. 23, 2021

(54) USE OF NATURAL-ABUNDANCE STABLE ISOTOPES AND DNA GENOTYPING FOR IDENTIFYING BIOLOGICAL PRODUCTS

(71) Applicant: ORITAIN GLOBAL LIMITED, Dunedin (NZ)

(72) Inventor: John P. JASPER, Niantic, CT (US)

(21) Appl. No.: 17/295,393

(22) PCT Filed: Nov. 15, 2019
(86) PCT No.: PCT/US2019/061745

§ 371 Date: May 19, 2021

Related U.S. Application Data
(60) Provisional application No. 62/769,939, filed on Nov. 20, 2018.

(51) Int. Cl. G16B 20/00

(52) U.S. Cl. CPC G16B 20/00

(57) ABSTRACT

The combined use of natural-abundance stable-isotopic analysis of bulk materials and DNA genotyping of biological materials is a highly-specific (˜1:1×10¹⁷) fingerprinting method for identifying such products in supply chains.

### FIELD OF THE INVENTION

A combined stable-isotopic and DNA genotyping method comprising a mathematical array of concentration ratios of isotopes found in a biological sample and coexisting genetic information from the DNA or RNA, said mathematical array being presented in a machine-readable form and comparable to analytical results whereby the sample can be distinguished from other similar samples, said machine readable form also being indexed through stored sample information. The stored sample information can be displayed when desired. By the combined stable isotopic and DNA identification of the invention, a sample can be securely traced through the supply chain for the manufacturing of a sample, marketing of a sample and the use of the sample.

### BACKGROUND

In ecology and genetics, a reaction norm, also called a norm of reaction, describes the pattern of phenotypic expression of a single genotype across a range of environments. One use of reaction norms is in describing how different species—especially related species—respond to varying environments. But differing genotypes within a single species can also show differing reaction norms relative to a particular phenotypic trait and environmental variable. For every genotype, phenotypic trait, and environmental variable, a different reaction norm can exist; in other words, an enormous complexity can exist in the interrelationships between genetic and environmental factors in determining traits.

Gene-environment interaction (or genotype-environmental interaction or G×E) is when two different genotypes respond to environmental variation in different ways. A norm of reaction is a graph that shows the relationship between genes and environmental factors when phenotypic differences are continuous. They can help illustrate G×E interactions. When the norm of reaction is not parallel, there is a gene by environment interaction. This indicates that each genotype responds to environmental variation in a different way. Environmental variation can be physical, chemical, biological, behavioral patterns or life events.

It is therefore seen that there is a need to provide methods for assessing gene-environment interactions, but current methods fail to provide the degree of specificity desired. The present invention addresses the shortcoming of current methodologies.

### SUMMARY OF THE INVENTION

The present invention relates to a method for objectively characterizing a biological sample containing a genetic, proteinaceous, catabolic, or metabolic constituent, comprising:

(a) obtaining isotopic data from elements present in said sample; providing a mathematical array that includes the isotopic data, the mathematical array being fixed in a readable form, said readable form with said mathematical array fixed thereon being an identification of said sample,
(b) obtaining genomic, proteinomic, catabolomic, or metabolomic data on the sample,
(c) constructing an integrated identifying data array from the isotopic data obtained in step
(a) and the genomic, proteinomic, catabolomic, or metabolomic data obtained in step (b), and
(d) providing an objective characterization of the biological sample.

In other embodiments, the present invention relates to a method according wherein the isotopic data does not include data obtained from a taggant.

In other embodiments, the present invention relates to a method wherein the elements are selected from elements that have two or more isotopes.

In other embodiments, the present invention relates to a method wherein the elements are selected from hydrogen, carbon, nitrogen, oxygen, sulfur, chlorine, and bromine, and combinations thereof.

In other embodiments, the present invention relates to a method wherein the isotopes are stable isotopes.

In other embodiments, the present invention relates to a method wherein the stable isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof.

In other embodiments, the present invention relates to a method according wherein the isotopes are selected from the following pairs of isotopes: ¹H and ²H, ¹²C and ¹³C, ¹⁴N and ¹⁵N, ¹⁶O and ¹⁸O, ³²S and ³⁴S, ³⁵Cl and ³⁷Cl, and ⁷⁹Br, and ⁸¹Br.

In other embodiments, the present invention relates to a method wherein the isotopes are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br.

In other embodiments, the present invention relates to a method wherein the isotopic data and the genomic, proteinomic, catabolomic, or metabolomic data is intrinsic data to the product.

In other embodiments, the present invention relates to a method wherein the integrated data (c) is fixed in a computer or machine-readable form.

In other embodiments, the present invention relates to a method wherein the biological product contains a genetic constituent and the genomic data is obtained by genotyping.

In other embodiments, the present invention relates to a method wherein the genetic constituent is selected from DNA, RNA, nucleotide fragments, and nucleic acids.

In other embodiments, the present invention relates to a method wherein the isotopic data is given with respect to a reference standard.

In other embodiments, the present invention relates to a data array for objectively characterizing a biological sample containing a genetic, proteinaceous, catabolic, or metabolic constituent, comprising:

(a) isotopic data from elements present in said sample; providing a mathematical array that includes the isotopic data, the mathematical array being fixed in a readable form, said readable form with said mathematical array fixed thereon being an identification of said sample, and
(b) genomic, proteinomic, catabolomic, or metabolomic data on the sample,
wherein the isotopic data of (a) and the genomic, proteinomic, catabolomic, or metabolomic data of (b) are integrated into an identifying data array for objectively characterizing the biological sample.

In other embodiments, the present invention relates to a data array wherein the elements are selected from elements that have two or more isotopes.

In other embodiments, the present invention relates to a data array wherein the elements are selected from hydrogen, carbon, nitrogen, oxygen, sulfur, chlorine, and bromine, and combinations thereof.

In other embodiments, the present invention relates to a data array wherein the isotopes are stable isotopes.

In other embodiments, the present invention relates to a data array wherein the stable isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof.

In other embodiments, the present invention relates to a data array wherein the isotopes are selected from the following pairs of isotopes: ¹H and ²H, ¹²C and ¹³C, ¹⁴N and ¹⁵N, ¹⁶O and ¹⁸O, ³²S and ³⁴S, ³⁵Cl and ³⁷Cl, and ⁷⁹Br, and ⁸¹Br.

In other embodiments, the present invention relates to a data array wherein the isotopes are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br.

In other embodiments, the present invention relates to a data array wherein the isotopic data and the genomic, proteinomic, catabolomic, or metabolomic data is intrinsic data to the product.

In other embodiments, the present invention relates to a data array wherein the integrated data is fixed in a computer or machine-readable form.

In other embodiments, the present invention relates to a data array wherein the biological product contains a genetic constituent and the genomic data is obtained by genotyping.

In other embodiments, the present invention relates to a data array wherein the genetic constituent is selected from DNA, RNA, nucleotide fragments, and nucleic acids.

In other embodiments, the present invention relates to a data array wherein the isotopic data is given with respect to a reference standard.

### BRIEF DESCRIPTION OF THE DRAWINGS

FIG. 1 shows a flow diagram of the G×E fingerprinting process of the present invention.

FIGS. 2A, 2B, and 2C illustrate three graphic representations of the G×E invention of the present invention for a typical biological analyte of a seed sample, to readily distinguish different plant seeds from different varieties grown under different conditions, e.g. different regions. FIG. 2A illustrates the same G, different E: same genetic make-up, different environment of growth/biosynthesis. FIG. 2B illustrates a different G, same E: different genetic make-up, same environment of growth/biosynthesis. FIG. 2C illustrates the same and different G's and E's: both the same and different genetic make-up, and both the same and different environment of growth/biosynthesis.

FIG. 3 shows the statistical distribution of data from a G×E sample analysis as a two-dimensional plot represented as an elliptical distribution. The ellipse is indicated as “e” and the centroid of the ellipse is indicated as “c”.

FIG. 4 illustrates the bare axes used to construct a three-dimensional plot from a G×E sample analysis. In this example, the x-axis represents the isotopic composition of water, which is represented as the isotopic difference (δ₁) from an International Atomic Energy (IAEA) standard for hydrogen and oxygen of water δ₁, (H, O). The y-axis represents the isotopic composition of the bulk biomass for the sample (such as from the carbohydrates, proteins, lipids, and nucleic acids, etc. from the sample), which is represented as the isotopic difference (δ₂) from an IAEA standard for carbon, nitrogen, and sulfur δ₂, (C, N, S). The z-axis represents the genetic parameter (G) for the homology or difference of the genetic sample based on a 0 to 1 scale.

FIG. 5 illustrates a three-dimensional plot from a G×E sample analysis using the coordinate system illustrated in FIG. 4. The x-axis defines PC1 (the principal component 1) for deuterium and oxygen 18, PC1(δD, δ¹⁸O). The y-axis defines PC2 (the principal component 2) for carbon 13, and also alternatively with nitrogen 15 and/or sulfur 34, PC2 (δ¹³C, or alternatively with δ¹⁵N, and/or δ³⁴S). The z-axis represents the genetic parameter (G) for the homology or difference of the genetic sample based on a 0 to 1 scale. The plot shows an ellipsoid “e” with a vector “v” from the origin of the x, y, z coordinates to the center of the ellipsoid, i.e. the centroid “c”.

FIG. 6 illustrates a three-dimensional plot from a G×E sample analysis from FIG. 5 showing the projection of the ellipsoid onto each of the (x,y), (y,z), and (x,z) planes.

### DETAILED DESCRIPTION OF THE INVENTION

G×E: G (genetics)×E (environment) is a powerful and elegant concept to trace and authenticate biological materials—i.e. materials containing DNA or RNA (e.g., plant seeds such as corn, wheat, cotton, etc.) —relative to the environment via stable isotopes.

In present invention we assert that there is a G×E relationship for one genotype: For example, “Iowa” (genetic type) soybeans grown in Iowa will likely have a very different (C, H, O, N, S) isotopic composition than “Iowa” soybeans grown in China—same DNA, different isotopic signal. This difference could be presented in a variety of ways, such as an X-Y graph (DNA phenotype vs bulk isotopes).

Although the concept of G×E is generally known, the methods and data arrays of the present invention for determining and quantitating G×E are believed to be new. As is shown herein, the present methods and data sets allow for new and useful applications for authenticating a biological sample or authenticating and/or distinguishing between two or more biological samples. The present methods and data sets provide a powerful means for performing what was not able to be performed before. The present invention takes the unique combination and integration of genetic fingerprinting data, i.e. genomic or sequencing data, with high resolution isotope ratio mass spectrometry data to provide an integrated data array that is useful for the methods herein.

Isotope ratio mass spectrometry (IRMS) is a specialized branch of mass spectrometry utilizing the relative abundance of isotopes in a given sample. The methodology allows for the precise measurement of mixtures of naturally occurring isotopes. Most instruments used for such precise determination of isotope ratios are of the magnetic sector type. The field of IRMS is of interest because differences in mass between different isotopes leads to isotope fractionation. This fractionation results in measurable effects on the isotopic composition of samples, thus providing a window into their biological or physical history. Consider the following example. The hydrogen isotope, deuterium (D or ²H), has nearly double the mass of ordinary hydrogen (¹H). Consequently, there is a significant difference in the mass of an ordinary water H₂O molecule versus HDO (a water molecule in which one of the hydrogens is replaced by a deuterium). Processes involving the evaporation of water or the cleavage of hydrogen-water bonds or of the disassociation of hydrogen bonds between water and/or other molecules will exhibit a fractionation. Consequently, water sources in different locations around the earth will most likely have different, and thus distinguishing, isotopic ratios or “fingerprints” of D to H.

Isotope ratios are generally given with respect to a standard, which given a δ value allows calculation of a relative isotopic abundance. Reference standards can be found in Hayes, J. M., Practice and Principles of Isotopic Measurements in Organic Geochemistry, Revision 2, August 2002, pages 1-15, particularly Table 1, the gist of which is excerpted here, and the reference which is incorporated by reference herein in its entirety.

TABLE 1
Isotopic Compositions of Primary Standards

| Primary Standard | Isotope Ratio |
|---|---|
| Standard Mean Ocean Water (SMOW) | ²H/¹H |
| | ¹⁸O/¹⁶O |
| | ¹⁷O/¹⁶O |
| PeeDee Belemnite (PDB) | ¹³C/¹²C |
| | ¹⁸O/¹⁶O |
| | ¹⁷O/¹⁶O |
| Air (AIR) | ¹⁵N/¹⁴N |
| Canyon Diablo Trolite (CDT) | ³⁴S/³²S |

Nucleic acid sequencing such as DNA or RNA sequencing can be used to determine the sequence of individual genes (DNA) or of the genes encoding for RNA structures such as the 16S subunit of the ribosome, which is useful for genetic identification. The methodology is used to study genomes and the proteins they encode. The advent of relatively inexpensive and rapid sequencing methodologies has allowed for the determination of sequences of DNA and RNA from biological samples to allow for their identification. These methods have led to the field of genomics, which focuses on the structure, function, evolution mapping, and editing of the genome, i.e. the genetic material of an organism. In preparing a sample for sequencing, polymerase chain reaction (PCR) is used to make copies of a specific DNA segment. In other words, the DNA sequences are exponentially amplified to generate sufficient quantities of material for genetic sequencing.

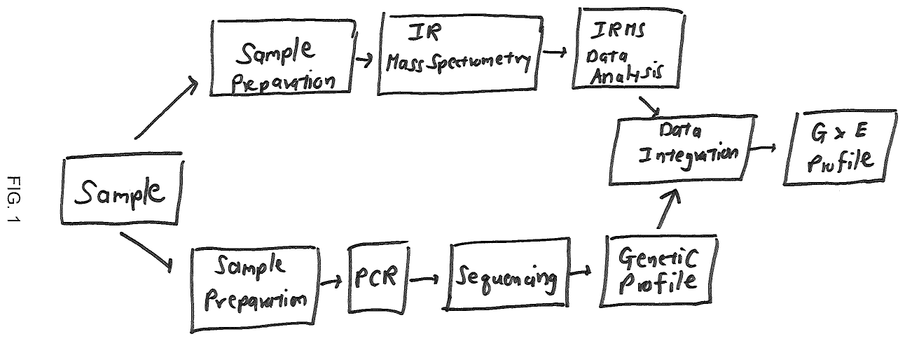

FIG. 1 shows a flow diagram of the G×E fingerprinting process of the present invention. A biological sample is partitioned down two different, parallel pathways. In one path, the sample is prepared for isotope ratio (IR) mass spectrometry analysis. This typically involves combustion of the sample into small molecules such as CO₂, N₂, SO₂, etc., which are then analyzed for their isotope ratios in an isotope ratio mass spectrometer. In the other path, the sample is prepared for genetic analysis. This can involve various extraction, purification, and concentrating protocols for isolating the DNA or RNA from the sample, followed by amplification processes such as PCR. The DNA or RNA material is then sequences using routine sequencing methodologies to genetically characterize the sample to provide a genetic profile. The data collected from the two separate analysis pathways is integrated or combined to produce a single array of the isotope ratio mass spectrometric data and the genetic data, or a G×E profile. As described below, the combination of the two data paths can be performed in different ways.

Relative Specificities of DNA versus Stable-Isotopic Analysis

| DNA Profiling | Stable-Isotopic Analysis |
|---|---|
| GMW: DNA, E. coli: ~3 × 10⁹ | GMW: Spans ~2 Daltons to 10⁹ Daltons |
| Specificity: ~1:10⁷ | Specificity of Light Isotopes |

Where GMW is (gram molecular weight)
(C, H, O, N, S)

| #δ | Dynamic Range | Specificity |
|---|---|---|
| 1 | 100¹ | 1:10² |
| 2 | 100² | 1:10⁴ |
| 3 | 100³ | 1:10⁶ |
| 4 | 100⁴ | 1:10⁸ |

The combination of DNA and natural stable isotopes (each with a specificity of ˜10⁷) is an exceptionally strong pairing, yielding very high specificity.

We assessed the compounded power of stable isotopes and human DNA. We estimated the specificity at ˜1 in 10¹⁷, extremely high. From our point of view, DNA is one among many compounds that we would typically isotopically fingerprint. Other biological compounds include RNA; proteins, peptides, and amino acids; products of catabolism; and metabolites.

We assessed the compounded power of bulk stable isotopes and bacterial DNA—DNA has a specificity of 3×10¹⁷. We estimated the specificity at ˜3 in 10¹⁷, which is extremely high for such assessments.

In the foregoing δ is a measure of the parts per thousand (per mil or “‰”) difference (either positive or negative) relative to an internationally accepted standard. For example, considering carbon 12 and carbon 13, δ¹³C is determined as:

δ¹³C═{[(¹³C/¹²C)ₛₐₘₚₗₑ/(¹³C/¹²C)_{standard}]−1}×1000‰.

Other isotopic ratios are similarly determined and calculated.

DNA is one among many compounds that we can isotopically fingerprint. We can therefore asses the bulk composition of a biological sample (e.g., wheat seeds, cotton) and separately a quantitative index of the coexisting DNA genotype. Stable-isotopic fingerprinting of a DNA molecule (as a bulk organic phase) and its genotype would then be an example of a focused application of our method. Typically, we can isotopically fingerprint a bulk material (e.g., a bulk wheat seed etc.) and a quantitative index of its DNA genotype. Although genomic identification, i.e. genotyping, of genetic material in the samples (e.g., DNA, RNA, nucleic acid fragments, nucleic acids) is an important application of this method, other biological materials can also be analyzed and identified for this purpose. For example, proteomics can be used to obtain identifying information on proteins, peptides, and amino acids in the samples. Catabolomics and metabolomics can be used to obtain identifying information on products of catabolism and metabolism in the samples. Similarly, other “omic” techniques and information can be obtained on other biological components.

DNA is a linear sequence of four base pairs of nucleotides (G—guanine, T—thymine, A—adenine, and C—cytosine) that encode genetic information. Also, involved is RNA, based on the nucleotides (G—guanine, U—uracil, A—adenine, and C—cytosine).

Natural abundance stable isotopes (e.g., C, H, O, N, S) record the isotopic provenance of biological materials with great specificity, as described above. In particular, the stable H and O isotopes of water record the environment (E) in which the material was biosynthesized. The C, N, and S isotopes record the isotopic composition of the biological material itself to provide a highly specific isotopic fingerprint.

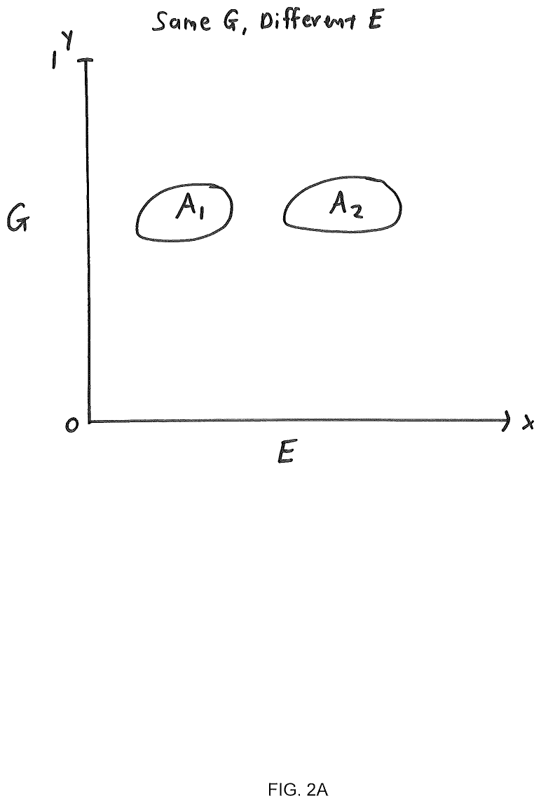

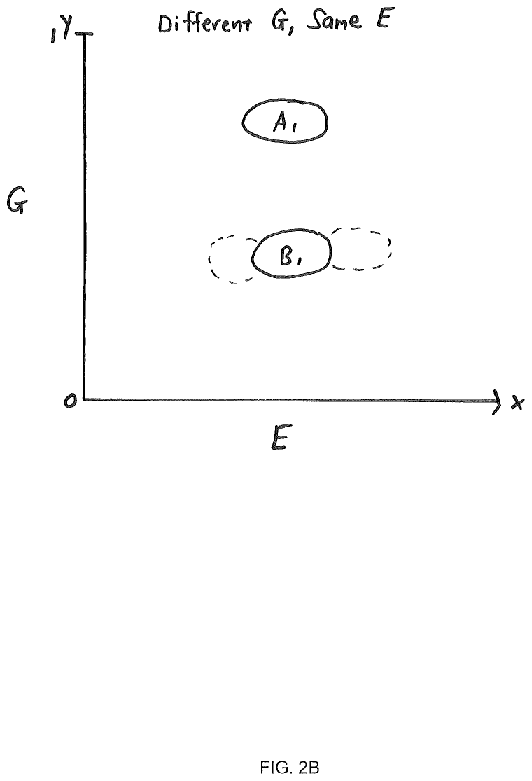

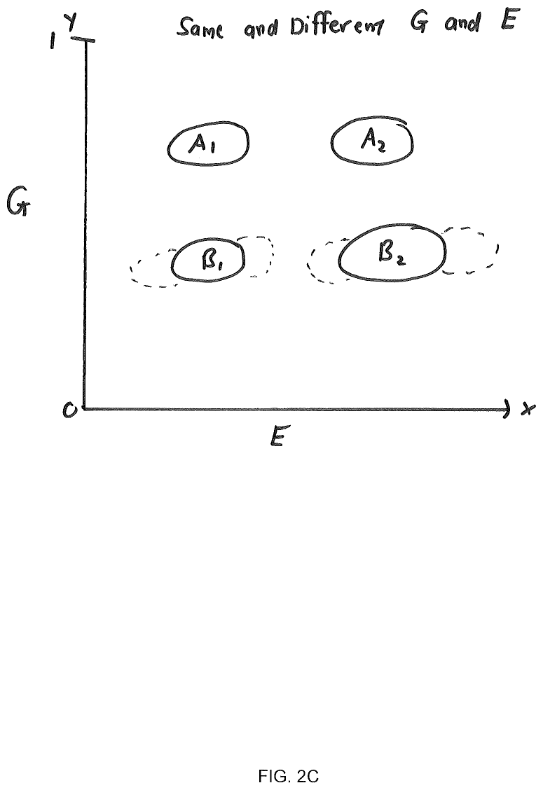

In an example, the G×E application can be shown on a bivariate plot (x,y-graph) as shown in FIGS. 2A, 2B, and 2C. In particular, three examples are given:

FIG. 2A illustrates the same genetic origin for the biological plant materials grown in different environments. In other words, the same G, but different E's. This result is illustrated by the expected elliptical range of data of A₁ and A₂ for the sample grown in two different environments.

FIG. 2B illustrates biological plant materials of different genetic origin grown in the same environment. In other words, different G's, but the same E. Even though the ellipses A₁ and B₁ are shown as directly above each other on the E axis, because of the elemental composition of the samples varying due to genetic differences, they would be expected to demonstrate isotopic differences not reliant upon the environment. This isotopic effect due to the genetics is illustrated by the dashed ellipses (offsets) shown in the figure that would be expected to be offset from the central ellipses. However, if there is an environmental effect, it would be expected to be much larger as shown in example 2C.

FIG. 2C illustrates the situation of biological plant materials of either the same or different origin grown in either the same or different environments. In other words, an array based on the same or different G's and the same or different E's. This situation is illustrated with the ellipses A1, B1, A2, and B2, wherein the dashed ellipses show possible offsets.

Parameterizing genetics: Linear DNA molecules are made up of two types of sequences: conserved and variable sections of DNA strands, all composed of four base pairs (ATCG or AUCG). These DNA sequences can subsequently be translated into or expressed as proteins.

With these sequences we can use the correlation coefficients (r²) of either DNA or protein as reference sections or locations. Correlation coefficients of these reference sections span from zero to 1, with r²=0 indicating no correlation and r²=1 indicating a perfect match.

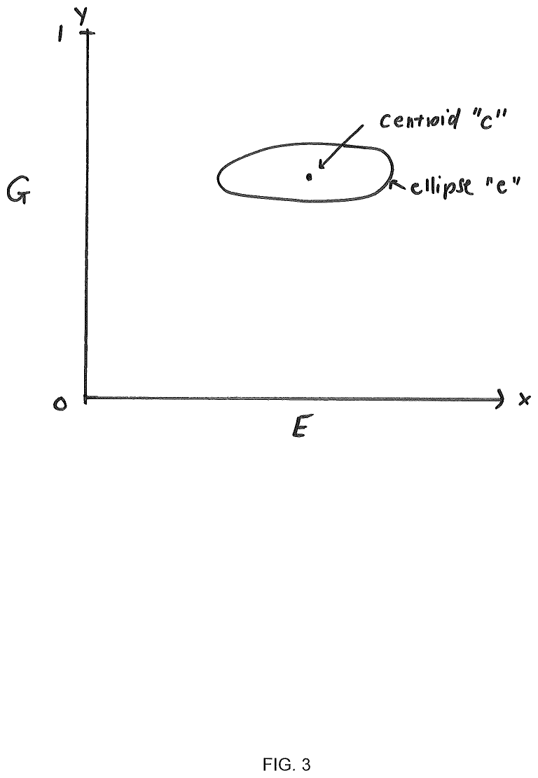

FIG. 3 shows the statistical distribution of data from a G×E sample analysis as a two-dimensional plot represented as an elliptical distribution (an ellipse “e”, with its centroid “c”). The size and shape of the ellipse will depend upon the statistical distribution of the sample. The centroid of the suite of data describes the statistical average across all data points in a given dimension.

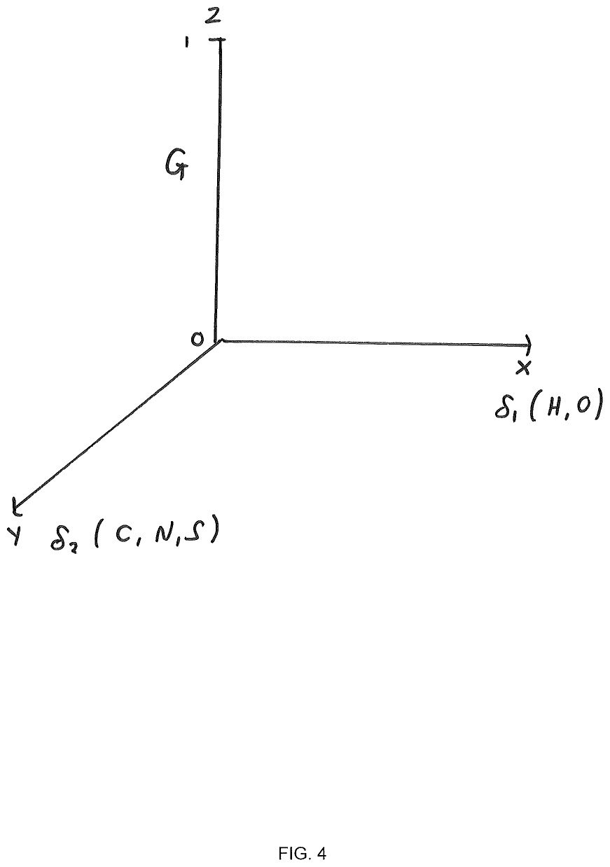

FIG. 4 illustrates the bare axes used to construct a three-dimensional plot from a G×E sample analysis. The x-axis represents the isotopic composition of water, which is represented as the isotopic difference (δ₁) from an international IAEA standard for hydrogen and oxygen of water δ₁, (H, O). This aspect is based on the water uptake of the samples and is highly dependent on the geographic and geochemical parameters, i.e. the location and biological conditions. The y-axis represents the isotopic composition of the bulk biomass for the sample (such as from the carbohydrates, proteins, lipids, and nucleic acids, etc. from the sample), which is represented as the isotopic difference (δ₂) from an IAEA standard for carbon, nitrogen, sulfur δ₂, (C, N, S). This aspect characterizes the isotopic composition of the biomass resulting from it having been produced under a given set of environmental conditions. The z-axis represents the genetic parameter (G) for the homology or difference of the genetic sample based on a 0 to 1 scale. This data is obtained using standard sequencing techniques and algorithms and calculations for assessing the genetic similarity or differences amongst the biological samples.

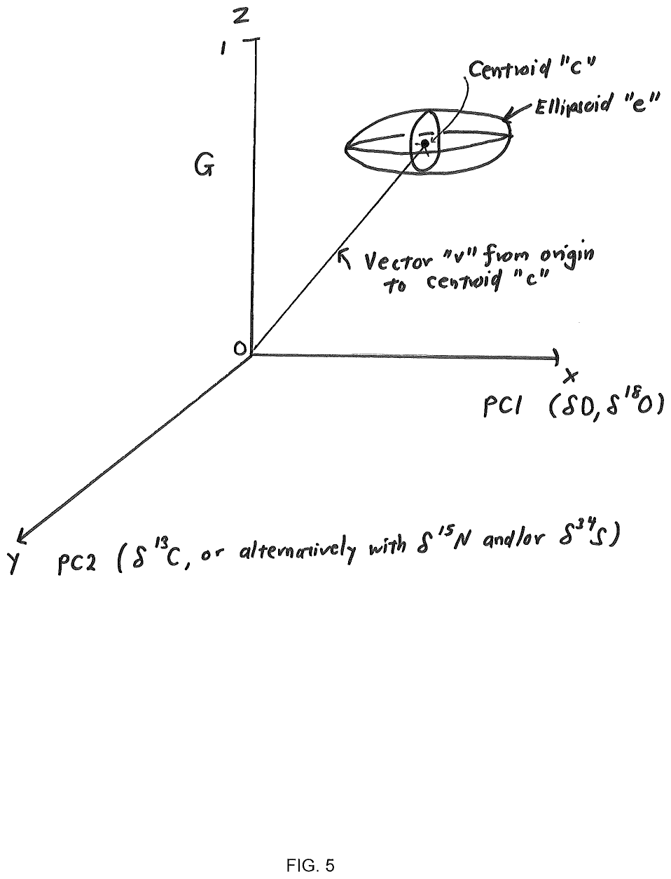

FIG. 5 illustrates a three-dimensional plot from a G×E sample analysis using the coordinate system illustrated in FIG. 4. The x-axis defines PC1 (the principal component 1) for deuterium and oxygen 18, PC1(δ D, δ ¹⁸O). The y-axis defines PC2 (the principal component 2) for carbon 13, and also alternatively with nitrogen 15 and/or sulfur 34, PC2 (δ ¹³C, δ ¹⁵N, δ ³⁴S). The z-axis represents the genetic parameter for the homology or difference of the genetic sample based on a 0 to 1 scale. The plot shows an ellipsoid “e” with a vector “v” from the origin of the x, y, z coordinates to the center of the ellipsoid, i.e. the centroid “c” The vector is useful when comparing the differences between or amongst two or more samples, each of which would have their own distinct ellipsoid.

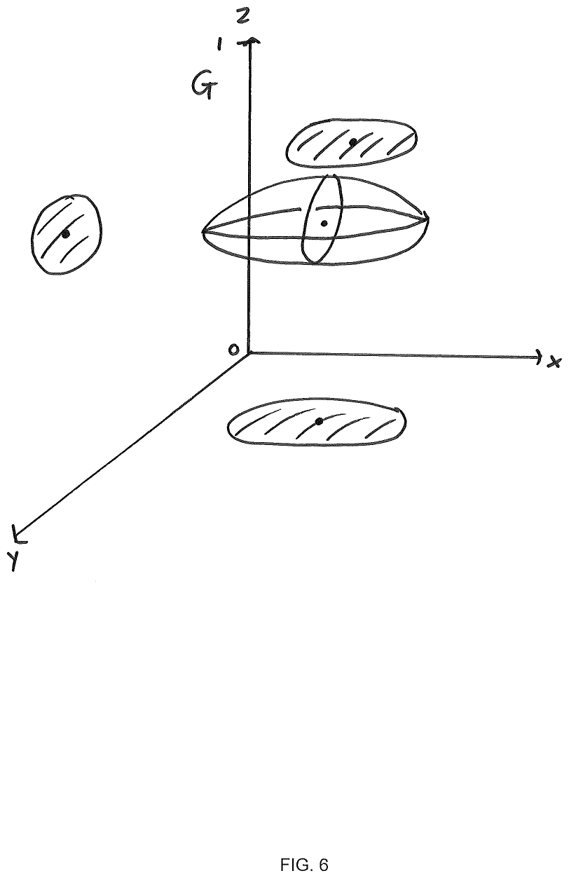

FIG. 6 illustrates a three-dimensional plot from a G×E sample analysis from FIG. 5 showing the projection of the three-dimensional ellipsoid onto each of the (x,y), (y,z), and (x,z) planes as two-dimensional ellipsoids. The purpose of the plot of FIG. 6 is to split out the data into two dimensional groups for easier visualization, interpretation, and analysis.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1

### The Same Wine Grape Grown in the Sonoma Valley and the Napa Valley

This is an example of genetically identical wine grape varieties being grown in two nearby, yet different geographic and climatic environments.

In this example the genetics (G) is constant, but the environmental conditions (E) under which the grapes are grown are different. The genetic profiling is expected to be identical, but the end product of the grapes should vary because of differences in the growing conditions such as soil, water, fertilizers, etc.

The data is expected to be as that shown in the generalized FIG. 2A.

### Example 2

### Two Different Wine Grapes Grown in the Sonoma Valley

This is an example of genetically different wine grape varieties being grown in the same geographic and climatic environments.

In this example the genetics (G) is different, but the environmental conditions (E) under which the grapes are grown are the same. The genetic profiling is expected to be different. The end product of the grapes should vary because of the isotopic differences of the compositions of the grapes superimposed upon the different growing conditions such as soil, water, fertilizers, etc. This environmental variation is expected to be expressed as larger isotopic differences that would be much greater than the isotopic differences due to the genetics.

The data is expected to be as that shown in the generalized FIG. 2B.

### Example 3

### Two Different Wine Grapes Grown in the Sonoma Valley and the Napa Valley

This is an example of genetically different wine grape varieties being grown in two nearby, yet different geographic and climatic environments.

In this example the genetics (G) is different and the environmental conditions (E) under which the grapes are grown are different. The genetic profiling is expected to be different. The end products of the grapes would also be different because of differences in the growing conditions such as soil, water, fertilizers, etc.

The data is expected to be as that shown in the generalized FIG. 2C.

### Example 4

The Same Seed Crop, e.g. Corn, Grown Using Two Different Nitrogen Source Fertilizers

This is an example of genetically identical corn varieties being grown on the same field with part of the field fertilized with a synthetic nitrogen source (Haber process synthetic fertilizer) and the other part of the filed fertilized with an organic nitrogen source (manure).

In this example the genetics (G) is constant, but the environmental conditions (E) under which the corn is grown are different. The genetic profiling is expected to be identical, but the end product of the corn should vary because of differences in the nitrogen source.

The data is expected to be as that shown in the generalized FIG. 2A.

### Example 5

### Arabica and Robusta Coffee Grown in Two Different Locations

In this example Arabian or Arabica coffee (Coffea arabica) and robusta coffee (Coffea canephora, also known as Coffea robusta) (two different G's) are grown in two different geographical locations, e.g. Brazil and Vietnam (two different E's). Arabica coffee is generally preferred as being of a better quality and having a better taste and aroma that robusta coffee, which is considered as inferior and is often described as more harsh and bitter. Arabica coffee beans generally sell at a premium of greater than 1.5 times the price of robusta coffee beans. Arabica beans comprise about 60 percent of world production with robusta beans comprising about 40 percent. It would therefore be desirable to identify a sample of coffee and its growing location to authenticate Arabica coffee grown in Brazil or Vietnam from robusta coffee grown in those same two locations. The present method would therefore distinguish Arabica coffee from robusta coffee grown in Brazil. This would be highly desirable to avoid robusta coffee grown in Vietnam as being misbranded as higher quality Arabica coffee grown in Brazil or robusta coffee grown in Brazil being misbranded as Brazilian Arabica coffee.

The data is expected to be as that shown in the generalized FIG. 2C.

### REFERENCES

U.S. Pat. No. 7,323,341 B1, Stable Isotopic Identification and Method For Identifying Products By Isotopic Concentration, to Jasper, issued Jan. 29, 2008.
U.S. Pat. No. 8,367,414 B2, Tracing Processes Between Precursors and Products By Utilizing Isotopic Relationships, to Jasper, issued Feb. 5, 2013.
PCT Application Publication No. WO2015/103183, Method for Continuously Monitoring Chemical or Biological Processes, to Jasper, published Jul. 9, 2015.
PCT Application Publication No. WO2016/109631, Isotopic Identification and Tracing of Biologic Products, to Jasper, published Nov. 12, 2015.
PCT Application Publication No. WO2015/103183, Molecular Isotopic Engineering, to Jasper published Jul. 7, 2016.
Hayes, J. M., Practice and Principles of Isotopic Measurements in Organic Geochemistry, Revision 2, August 2002, pages 1-15, particularly Table 1.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps or components, it is also contemplated that the methods and systems consist essentially of, or consist of, the recited steps or components. Furthermore, the order of steps or order for performing certain actions is immaterial as long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as composed of the components prior to mixing, because upon mixing certain components can further react or be transformed into additional materials.

All percentages and ratios used herein, unless otherwise indicated, are by weight.

What is claimed is:

1. A method for objectively characterizing a biological sample containing a genetic, proteinaceous, catabolic, or metabolic constituent, comprising:
(a) obtaining isotopic data from elements present in said sample; providing a mathematical array that includes the isotopic data, the mathematical array being fixed in a readable form, said readable form with said mathematical array fixed thereon being an identification of said sample,
(b) obtaining genomic, proteinomic, catabolomic, or metabolomic data on the sample,
(c) constructing an integrated identifying data array from the isotopic data obtained in step (a) and the genomic, proteinomic, catabolomic, or metabolomic data obtained in step (b), and
(d) providing an objective characterization of the biological sample.

2. A method according to claim 1 wherein the isotopic data does not include data obtained from a taggant.

3. A method according to claim 1 or 2 wherein the elements are selected from elements that have two or more isotopes.

4. A method according to any of claims 1 to 3 wherein the elements are selected from hydrogen, carbon, nitrogen, oxygen, sulfur, chlorine, and bromine, and combinations thereof.

5. A method according to claim 3 wherein the isotopes are stable isotopes.

6. A method according to claim 5 wherein the stable isotopes are selected from ¹H, ²H, ¹²C, ¹³C, ¹⁴N, ¹⁵N, ¹⁶O, ¹⁸O, ³²S, ³⁴S, ³⁶Cl, ³⁷Cl, ⁷⁹Br, and ⁸¹Br and combinations thereof.

7. A method according to claim 6 wherein the isotopes are selected from the following pairs of isotopes: ¹H and ²H, ¹²C and ¹³C, ¹⁴N and ¹⁵N, ¹⁶O and ¹⁸O, ³²S and ³⁴S, ³⁵Cl and ³⁷Cl, and ⁷⁹Br, and ⁸¹Br.

8. A method according to claim 6 wherein the isotopes are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br.

9. A method according to any of claims 1 to 8 wherein the isotopic data and the genomic, proteinomic, catabolomic, or metabolomic data is intrinsic data to the sample.

10. A method according to any of claims 1 to 9 wherein the integrated data (c) is fixed in a computer or machine-readable form.

11. A method according to claim 1 wherein the biological sample contains a genetic constituent and the genomic data is obtained by genotyping.

12. A method according to claim 11 wherein the genetic constituent is selected from DNA, RNA, nucleotide fragments, and nucleic acids.

13. A method according to claim 1 wherein the isotopic data is given with respect to a reference standard.

14. A data array for objectively characterizing a biological sample containing a genetic, proteinaceous, catabolic, or metabolic constituent, comprising:
(a) isotopic data from elements present in said sample; providing a mathematical array that includes the isotopic data, the mathematical array being fixed in a readable form, said readable form with said mathematical array fixed thereon being an identification of said sample, and
(b) genomic, proteinomic, catabolomic, or metabolomic data on the sample,
wherein the isotopic data of (a) and the genomic, proteinomic, catabolomic, or metabolomic data of (b) are integrated into an identifying data array for objectively characterizing the biological sample.

15. A data array according to claim 14 wherein the elements are selected from elements that have two or more isotopes.

16. A data array according to claim 15 or 16 wherein the elements are selected from hydrogen, carbon, nitrogen, oxygen, sulfur, chlorine, and bromine, and combinations thereof.

17. A data array according to claim 15 wherein the isotopes are stable isotopes.

18. A data array according to claim 17 wherein the stable isotopes are selected from ¹H, ²H, ¹²C, ¹³C, ¹⁴N, ¹⁵N, ¹⁶O, ¹⁸O, ³²S, ³⁴S, ³⁵Cl, ³⁷Cl, ⁷⁹Br, and ⁸¹Br and combinations thereof.

19. A data array according to claim 18 wherein the isotopes are selected from the following pairs of isotopes: ¹H and ²H, ¹²C and ¹³C, ¹⁴N and ¹⁵N, ¹⁶O and ¹⁸O, ³²S and ³⁴S, ³⁵Cl and ³⁷Cl, and ⁷⁹Br, and ⁸¹Br.

20. A data array according to claim 18 wherein the isotopes are selected from the following isotope ratios: ²H/¹H, ¹³C/¹²C, ¹⁵N/¹⁴N, ¹⁸O/¹⁶O, ³⁴S/³²S, ³⁷Cl/³⁵Cl, and ⁸¹Br/⁷⁹Br.

21. A data array according to any of claims 14 to 20 wherein the isotopic data and the genomic, proteinomic, catabolomic, or metabolomic data is intrinsic data to the sample.

22. A data array according to any of claims 14 to 20 wherein the integrated data is fixed in a computer or machine-readable form.

23. A data array according to claim 14 wherein the biological sample contains a genetic constituent and the genomic data is obtained by genotyping.

24. A data array according to claim 14 wherein the genetic constituent is selected from DNA, RNA, nucleotide fragments, and nucleic acids.

25. A data array according to claim 14 wherein the isotopic data is given with respect to a reference standard.
